(19) United States
(12) Patent Application Publication
(10) Pub. No.: US 2014/0162950 A1
(43) Pub. Date: Jun. 12, 2014

(54) METHOD FOR INHIBITING PERITONEAL METASTASIS CAUSED BY GASTRIC CANCER CELLS

(71) Applicants: Cheng-Chi Chang, Taipei City (TW); Chiung-Nien Chen, Taipei City (TW); Hsin-Yu Li, Taipei City (TW); Min-Liang Kuo, Taipei City (TW)

(72) Inventors: Cheng-Chi Chang, Taipei City (TW); Chiung-Nien Chen, Taipei City (TW); Hsin-Yu Li, Taipei City (TW); Min-Liang Kuo, Taipei City (TW)

(73) Assignee: NATIONAL TAIWAN UNIVERSITY, Taipei City (TW)

(21) Appl. No.: 13/709,534

(22) Filed: Dec. 10, 2012

(51) Int. Cl. A61K 38/18

(52) U.S. Cl. CPC A61K 38/18
USPC 514/7.6; 435/7.1

(57) ABSTRACT

The present invention provides a method for inhibiting peritoneal metastasis caused by gastric cancer cells in a subject in need thereof comprising administering to the subject a pharmaceutically effective amount of a connective tissue growth factor (CTGF), wherein the CTGF binds to an integrin α3β1 of the gastric cancer cells. The present invention also provides a method for predicting peritoneal metastasis caused by gastric cancer cells in a subject comprising providing a peritoneal tissue from the subject; measuring a first expression amount of a connective tissue growth factor (CTGF) from the peritoneal tissue; and comparing the first expression amount to a reference expression amount of the CTGF from a non-peritoneal metastasis gastric cancer tissue.

### FIELD OF THE INVENTION

The present invention is related to a method for inhibiting peritoneal metastasis caused by gastric cancer cells.

### BACKGROUND OF THE INVENTION

Gastric cancer is the second leading cause of cancer related deaths worldwide and is the fifth leading cause of cancer related deaths in Taiwan. Peritoneal dissemination is one of the non-curative factors in gastric cancer and many efforts have been made to prevent this condition with limited success (Ann Surg Oncol 2009; 16(12):3217-8). Extensive metastasis to the lymph nodes and liver are also important factors in the poor prognosis of gastric cancer, but with peritoneal dissemination as the major contributing factor through direct spillage of cancer cells during surgery (J Surg Oncol 1992; 51(2):104-8). Development of peritoneal metastasis is a multistep process that begins with the detachment of cancer cells from primary tumor, forming circulating tumor cells (CTC), attachment of CTC to peritoneal mesothelial cells, retraction of mesothelial cells to expose basement membrane, attachment to basement membrane, degradation in the extracellular matrix, proliferation of cancer cells, and finally angiogenesis. Formation of CTC has been a clinically significant factor in peritoneal dissemination and prognosis, therefore contributes to cancer progression in patients with gastrointestinal cancers. Since peritoneal metastasis is one of the most frequent causes of non-curative surgery in gastric cancer therapy, it is important to prevent cancer cell adhesion to peritoneum. No effective method of prevention has been found. Therefore, developing a new therapeutic method for this mode of metastasis is very important.

It has previously demonstrated that calreticulin (CRT), a multifunctional protein that plays important roles in calcium regulation in the endoplasmic reticulum, and is a prognostic marker in gastric cancer contributing to angiogenesis, lymph node metastasis and survival in human gastric cancer. From microarray analysis, it has demonstrated that CRT expression level is reciprocal to the expression level of an important growth factor, connective tissue growth factor (CTGF). CTGF have been found to play diverse roles in a variety of cancers and known to be an important regulator for cell adhesion. Therefore, it is hypothesized that CTGF also plays an important role in gastric cancer.

CTGF is a secretory protein first discovered in 1991 (J Cell Biol 1991; 114(6):1285-94) and belongs to the CCN family. It is a multifunctional growth factor involved in wound healing, inflammation, cell adhesion, chemotaxis, apoptosis, tumor growth, and fibrosis (Angiogenesis 2002; 5(3):153-65). Elevated CTGF expression has been detected in various tumors. Additionally, CTGF can promote angiogenesis by regulating endothelial cell growth, migration, adhesion, and survival. However, recent studies have shown that over expression of CTGF in human oral squamous cell carcinoma (OSCC) reduces cell growth, tumorigenecity as well as OSCC invasion (Oncogene 2012; 31: 2401-2411). Similar tumor growth inhibitory effects were observed in lung cancer cells in which CTGF over expression was less angiogenic and metastatic due to blocking of the VEGF A signaling pathway (FASEB J 2002; 16(4219-24 CTGF was also reported to be a key regulator of colorectal cancer invasion and metastasis, and it appears to be a good prognostic factor (Gastroenterology 2005; 128(1):9-23). Furthermore, it has been found that recombinant CTGF is a potential therapeutic agent in colorectal cancer therapy (Clin Cancer Res 2011; 17(10):3077-88). On the other hand, CTGF has been reported to be an indicator for tumor progression in pancreatic cancer cells where elevated levels of CTGF expression has been demonstrated. Recent studies have also shown that CTGF is over-expressed in papillary thyroid carcinoma, promotes grown of papillary thyroid cancer cells, and is correlated with increased angiogenesis and migration in breast cancer cells (Int J Oncol 2011; 38(6):1741-7). These results suggest that CTGF have diverse functions in different types of cancers, and its exact mechanisms and functions in gastric cancer have not yet been clarified.

### BRIEF DESCRIPTION OF THE DRAWINGS

FIG. 1 shows the immunohistochemical staining of CTGF protein expression in human normal gastric tissues and gastric cancer tissues and Kaplan-Meier survival probability of CTGF positive and negative patients. Higher expression of CTGF was observed in normal gastric tissues and higher survival probability was observed in CTGF positive patients (A) compared to paired diffuse type (B) and paired intestinal type gastric cancer tissues (C). A: ×200 original magnification. B and C: ×400 original magnification. (D) Survival probability of CTGF positive (n=69) and CTGF negative (n=38) gastric cancer patients at 144 months post surgery (p=0.0014).

FIG. 2 shows the adhesion of gastric cancer cells in vitro and in vivo that is affected by CTGF expression level. (A) Wild-type AGS, N87 and MKN45 gastric cancer cells. Higher CTGF mRNA and protein level of AGS and N87 cells significantly reduced adhesion in vitro while lower CTGF mRNA and protein of MKN45 have significantly higher adhesion in vitro (p=0.043 for AGS v.s MKN45; p=0.015 for N87 v.s MKN45). (B) CTGF over-expression in MKN45 gastric cancer cells significantly inhibited adhesion in vitro compared to pCDNA3 control cells (p<0.05). (C) Lentivirus CTGF knockdown in AGS gastric cancer cells elevated adhesion in vitro (p<0.05). (D) SCID mice ip inoculated with MKN45/neo or MKN45/CTGF cells. In vivo tumorigenicity has shown that the number of tumor nodules is significantly decreased in MKN45/CTGF inoculated group (p<0.05).

FIG. 3 shows the gastric cancer cell adheres to fibronectin and laminin and CTGF inhibits adhesion via blocking integrin α3β1. (A) CTGF over-expression significantly inhibited MKN45 cells to fibronectin and laminin substratum in vitro compared to wild-type MKN45 gastric cancer cells. (B) integrin α3 and β1 blocking antibodies significantly inhibited MKN45 gastric cancer cell adhesion to laminin substratum. (C) Integrin α3 subunit blocking antibody inhibits CTGF knockdown AGS gastric cancer cell adhesion to laminin. (D) MKN45 cells significantly adhere to recombinant CTGF coated plate and adhesion is inhibited by integrin α3 blocking antibody. (E) Co-immunoprecipitation using integrin α3 antibody as IP antibody followed by detection using CTGF antibody showed that CTGF and integrin α3 bind to each other in vitro.

FIG. 4 shows the recombinant CTGF protein effectively blocks gastric cancer cell adhesion in vitro and in vivo as well as increases survival probability in vivo. (A) 0, 50, 100, and 200 ng/ml recombinant CTGF treated cells showed decreased adhesion ability at 6 hours post treatment in vitro. (p<0.01) (B) Recombinant CTGF treatment does not cause cell death at 0, 50, 100 and 200 ng/ml at 6 hours post treatment. (C) Recombinant CTGF treatment in mice showed decreased number of tumor nodules on the mesentery compared to non-treated mice. (D) Recombinant CTGF treated mice have significantly higher survival probability than non-treated mice. (P=0.0056). NS=no significance.

### SUMMARY OF THE INVENTION

The present invention provides a method for inhibiting peritoneal metastasis caused by gastric cancer cells in a subject in need thereof comprising administering to the subject a pharmaceutically effective amount of a connective tissue growth factor (CTGF), wherein the CTGF binds to an integrin α3β1 of the gastric cancer cells. The present invention also provides a method for predicting peritoneal metastasis caused by gastric cancer cells in a subject comprising providing a peritoneal tissue from the subject; measuring a first expression amount of a connective tissue growth factor (CTGF) from the peritoneal tissue; and comparing the first expression amount to a reference expression amount of the CTGF from a non-peritoneal metastasis gastric cancer tissue.

### DETAIL DESCRIPTION OF THE INVENTION

Unless otherwise specified, “a” or “an” means “one or ore”.

Connective tissue growth factor (CTGF) has diverse cellular functions and has been implicated in tumor development and progression. The aim of this invention is to investigate the roles of CTGF in peritoneal metastasis in gastric cancer (GC) as well as the underlying mechanism and its role in prognosis of post surgery gastric cancer patients.

Quantitative PCR and Western blot were performed to determine CTGF expression level. Gastric cancer cell lines that stably overexpressed or knockdown CTGF were generated for in vitro matrigel adhesion assay and in vivo peritoneal metastasis assay. Univariate, multivariate analysis, immunohistochemistry and survival probability were analyzed in gastric cancer patient after surgery. Using ECM-coated plates and integrin functional blocking antibodies, the specific ECM components involved in CTGF-regulated adhesion were screened and determined. Recombinant CTGF was directly added to cells in vitro Or co-inoculated with gastric cancer cells to SCID mice to evaluate its potential as a therapeutic agent to prevent peritoneal metastasis.

CTGF over-expression and recombinant protein treatment significantly inhibit cell adhesion. Furthermore, silenced endogenous CTGF expression by shCTGF plasmids enhanced adhesion ability in gastric cancer cells. In vivo peritoneal metastasis SCID animal model demonstrated that CTGF stable transfectants markedly decreased the numbers and size of tumor nodules in mesentery, compared to vector control in MKN45 cells. Clinically, statistical analysis of gastric cancer patient data showed that patients expressed higher CTGF levels have earlier TNM staging as well as higher survival probability post surgery. At the molecular level, it was found that integrin α3β1 was determined as the cell adhesion molecule mediating gastric cancer cell adhesion to laminin. In addition, blocking integrin α3β1 prevented gastric cancer cell adhesion to recombinant CTGF and immunoprecipitation have indicated that CTGF binds to integrin α3β1. Furthermore, co-inoculation of recombinant CTGF and gastric cancer cell lines in SCID mice demonstrated that rCTGF effectively inhibited peritoneal dissemination and significantly increased survival probability.

The results indicated that gastric cancer peritoneal metastasis is mediated through cell surface integrin α3β1 binding to laminin and CTGF effectively blocks the interaction by binding to integrin α3β1. Recombinant CTGF therefore demonstrated its therapeutic potential in prevention of gastric cancer peritoneal metastasis. Herein it was clearly demonstrated that CTGF blocking integrin adhesion to extracellular matrix in gastric cancer cell without affecting cell proliferation; therefore pose little cytotoxicity which is an important factor in therapeutics.

Therefore, the present invention provides a method for inhibiting peritoneal metastasis caused by gastric cancer cells in a subject in need thereof comprising administering to the subject a pharmaceutically effective amount of a connective tissue growth factor (CTGF), wherein the CTGF binds to an integrin ON of the gastric cancer cells.

In the preferred embodiment of the present invention, the CTGF is selected from a CTGF native protein (SEQ ID NO: 3), a CTGF recombinant protein or a C terminal (CT) domain of the CTGF (SEQ ID NO:4).

In the present invention, the inhibition of the peritoneal metastasis is due to the binding of the CTGF to the integrin α3β1 of the gastric cancer cells that blocks the gastric cancer cells bind to a laminin of a peritoneal cell.

In the preferred embodiment of the present invention, the subject is a mammal, and in the more preferred embodiment of the present invention, the mammal is a human.

The present invention also provides a method for prognosing peritoneal metastasis caused by gastric cancer cells in a subject comprising
(a) providing a peritoneal tissue from the subject;
(b) measuring a first expression amount of a connective tissue growth factor (CTGF) from the peritoneal tissue; and
(c) comparing the first expression amount to a reference expression amount of the CTGF from a non-peritoneal metastasis gastric cancer tissue,
   wherein a decrease in the first expression amount relative to the reference expression amount means the decrease of the CTGF that binds to an integrin 001 of the gastric cancer cells and is indicative that the subject is susceptible to peritoneal metastasis.

In the preferred embodiment of the present invention, the subject is a mammal, and in the more preferred embodiment of the present invention, the mammal is a human.

### EXAMPLES

The examples below are non-limiting and are merely representative of various aspects and features of the present invention.

### Patients

For CTGF expression profiling and survival probability analysis, a total of 107 patients with gastric cancer, who had undergone radical gastrectomy at National Taiwan University Hospital from January 1995 to September 2004, were included in this invention. They were 70 men and 37 women at the average age of 63.8 years. They were staged according to TNM system based on postoperative pathological reports. Criteria for consideration as curative resection were the complete removal of a primary gastric tumor, D2 dissection of regional lymph nodes, and no macroscopic tumor being left behind. They had no detectable metastasis in liver, peritoneum and distant organ at the time of surgery. No other previous or concomitant primary cancer was present. No patient had received chemotherapy and radiotherapy before surgery. Clinico-pathologic factors including age, sex, gross types of tumors (Borrmann classification), histological types of tumors (Lauren classification), depth of tumor invasion, status of lymph node metastasis, vascular invasion, and Helicobacter pylori (H. pylori) infection documented with histological findings were reviewed and stored in patients' data base. Vascular invasion was considered to be definite only when tumor cells and red blood cells were noted together in an endothelium-lined vascular space or when tumor cells were found in an endothelium-lined vascular space with a definite smooth muscle layer. The tissues were considered positive for H. pylori if faintly blue staining curved bacilli were seen in the mucus of crypts just adjacent to tumor using hematoxylin and eosin stain. The patients were followed up from 6 to 143 months after surgery. The follow-up intervals were calculated as survival intervals after surgery.

### Example 1

### CTGF Expression Profiling and Clinical Correlation Using Immunohistochemistry Staining of Gastric Cancer Patient Tissues

To investigate the localization of CTGF expression in gastric cancer tissue, the CTGF protein expression in normal and tumor specimens were stained immunohistochemically. The CTGF protein (SEQ ID NO: 3) was detected on formalin-fixed, paraffin-embedded section using the labeled streptavidine-biotin (LSAB) method after antigen retrieval. Briefly, deparaffinized sections were heated in a pressure cooker. After blocking with 3% H₂O, and non-immune horse serum, the slides were allowed to react with a monoclonal antibody (Transduction Laboratories, Lexington, Ky.) against human CTGF at a dilution of 1:40 at 4° C. overnight. The slides were incubated with link antibodies, followed by peroxidase conjugated streptavidin complex (LSAB kit, DAKO Corporation, Carpinteria, Calif.). The peroxidase activity was visualized with Diaminobenzidine tetrahydroxychloride (DAB, DAKO Corporation, Carpinteria, Calif.) as the substrate. The sections were lightly counterstained with hematoxylin. The results of immunohistological staining were classified using extent of cell stained; these were level 0 (negative staining), level 1 (<5% of tumor cells stained), level 2 (<50% of tumor cells stained), and level 3 (>50% of tumor cells stained). Levels 0 and 1 were grouped as low CTGF expression and levels 2 and 3 as high CTGF expression.

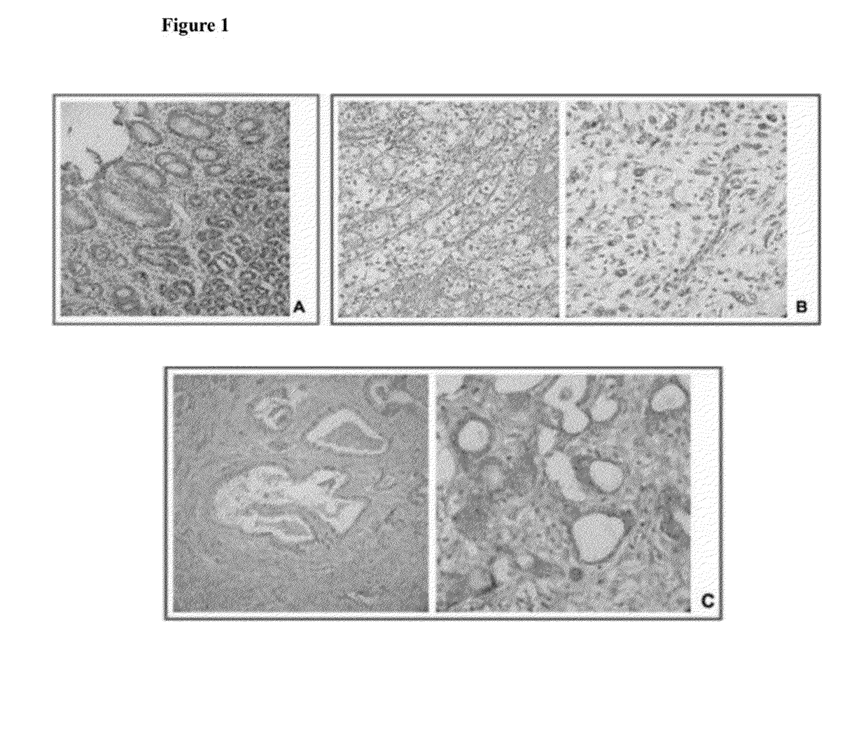

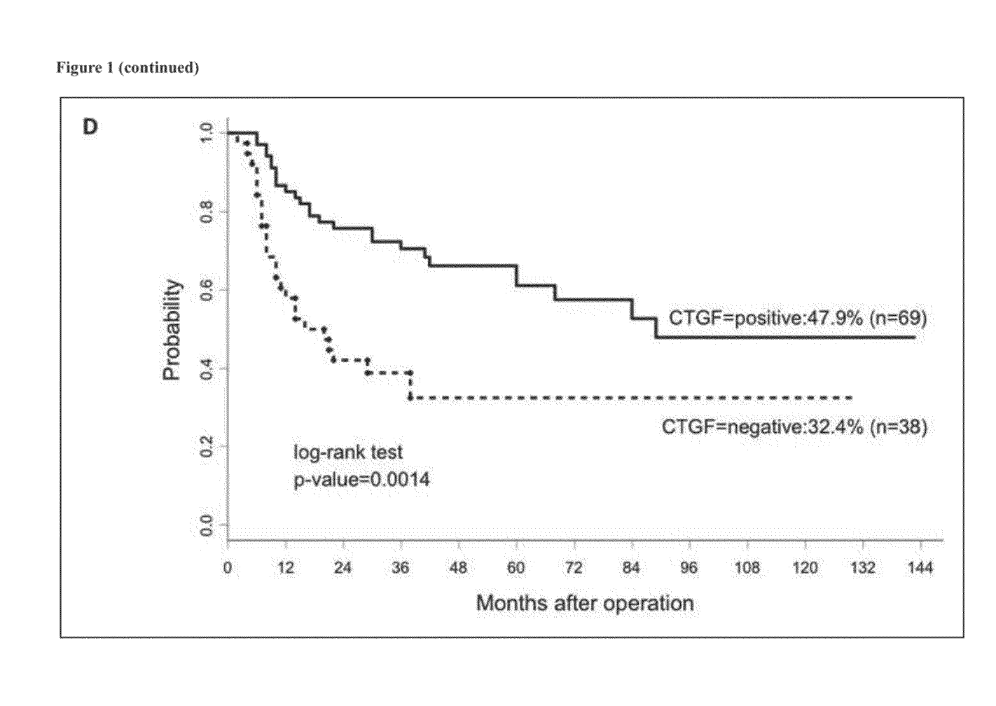

CTGF protein expression was high in normal gastric epithelium and moderate in diffuse type or intestinal type gastric adenocarcinoma tissues (FIG. 1A-C). Higher expression of CTGF was observed in normal gastric tissues and higher survival probability was observed in CTGF positive patients (A) compared to paired diffuse type (B) and paired intestinal type gastric cancer tissues (C). A: ×200 original magnification. B and C: ×400 original magnification. In tumor tissues, CTGF protein was predominantly localized in the cytoplasm. In studying tissue samples from 107 patients who underwent gastrectomy for gastric cancer, CTGF could be positively stained in 64% (69/107) of gastric cancer specimens.

The correlations between the expression of CTGF and the clinicopathological factors were shown in Table 1. A significant correlation was found between CTGF expression and peritoneal metastasis (p=0.000), and stage (p=0.037) by univariate analysis.

TABLE 1
Relationship between the expression of CTGF and
clinicopathological factors in 107 patients with gastric cancer

| | CTGF (−) | CTGF (+) | p Value |
|---|---|---|---|
| Age | 63.71 ± 11.91 | 63.86 ± 12.36 | 0.059 |
| Sex | | | 1 |
| Men | 25 | 45 | |
| Women | 13 | 24 | |
| Depth of tumor invasion (T) | | | 0.62 |
| T1 | 5 | 15 | |
| T2 | 6 | 12 | |
| T3 | 25 | 37 | |
| T4 | 2 | 5 | |
| Nodal status (N) | | | 0.352 |
| Node negative | 10 | 28 | |
| Node positive | 28 | 41 | |
| Gross tumor morphology | | | 0.349 |
| Early cancer | 4 | 14 | |
| Borrmann type I | 2 | 1 | |
| Borrmann type II | 5 | 11 | |
| Borrmann type III | 26 | 38 | |
| Borrmann type IV | 1 | 5 | |
| Lauren classification | | | 0.843 |
| Intestinal type | 16 | 30 | |
| Diffuse type | 18 | 34 | |
| Mixed type | 4 | 5 | |
| Vascular invasion | | | 0.306 |
| Negative | 13 | 32 | |
| Positive | 25 | 37 | |
| Peritoneal metastasis | | | 0 |
| Negative | 13 | 50 | |
| Positive | 25 | 19 | |
| Stages | | | 0.037 |
| I | 8 | 19 | |
| II | 3 | 12 | |
| III | 15 | 31 | |
| IV | 12 | 7 | |

Multivariate analysis have shown that only CTGF was significantly correlated with peritoneal metastasis (v0.002) (Table 2). Patients with high CTGF expression had better prognosis than those with low expression. In addition to serosal invasion, lymph node metastasis, vascular invasion, Borrmann type, and Lauren classification, CTGF protein expression was also an independent prognostic factors using multivariate analysis (Table 3).

TABLE 2
Expression of connective tissue growth factor and
clinicopathological factors: multivariate analysis

| Variable | B | SE | Exp(β) | Sig | odds ratio |
|---|---|---|---|---|---|
| Age | −0.015 | 0.02 | 0.54 | 0.462 | 0.985 |
| Sex | 0.094 | 0.486 | 0.038 | 0.846 | 1.099 |
| Borrmann types | −0.081 | 0.305 | 0.071 | 0.789 | 0.922 |
| Lauren classifiation | 0.469 | 0.412 | 1.296 | 0.255 | 1.598 |
| Serosal invasion | 0.487 | 0.472 | 1.065 | 0.302 | 1.627 |
| Lymph node metastasis | −0.170 | 0.377 | 0.202 | 0.653 | 0.844 |
| Distant metastasis | −0.720 | 0.673 | 1.145 | 0.285 | 0.487 |
| Vascular invasion | 0.206 | 0.612 | 0.114 | 0.736 | 1.229 |
| Peritoneal metastasis | −2.01 | 0.640 | 9.865 | 0.002 | 0.134 |

B, β regression coefficient;
SE, standard error;
Exp(β), exponent β;
Sig, significance (P value)

TABLE 3
Clinicopathological factors affecting survival rate: multivariate
analysis

| Variable | B | SE | Exp(β) | Sig | odds ratio |
|---|---|---|---|---|---|
| Age | −0.002 | 0.013 | 0.032 | 0.859 | 0.998 |
| Sex | 0.056 | 0.303 | 0.034 | 0.854 | 1.058 |
| Borrmann types | 0.659 | 0.290 | 5.143 | 0.023 | 1.932 |
| Lauren classifiation | 0.561 | 0.251 | 5.009 | 0.025 | 1.752 |
| Serosal invasion | 1.021 | 0.493 | 4.290 | 0.038 | 2.777 |
| Lymph node metastasis | 1.467 | 0.535 | 7.519 | 0.006 | 4.336 |
| Vascular invasion | 0.818 | 0.431 | 3.603 | 0.058 | 2.265 |
| CTGF | −0.720 | 0.288 | 6.244 | 0.012 | 0.487 |

B, β regression coefficient;
SE, standard error;
Exp(β), exponent β;
Sig, significance (P value)

Survival probability was calculated using the Kaplan-Meire survival probability test and patients who express higher level of CTGF were found to have higher survival probability post surgery (p=0.0014) (FIG. 1D). This strongly suggests that CTGF expression correlates with survival of gastric cancer patients and acts as a prognostic marker for patients who have undergone gastrectomy. This result also further determines the correlation of peritoneal dissemination and survival probability to CTGF expression level. This is an indication that peritoneal dissemination is modulated by CTGF.

### Example 2

### CTGF Expression and Adhesion Ability

To determine CTGF expression in wild-type gastric cancer cells, the human gastric cancer cell lines MKN45, N87 and AGS were grown in RPMI 1640 medium (Biological Industries, Israel) supplemented with 10% fetal bovine serum and penicillin/streptomycin/amphotericin B antibiotic cocktail (Biological Industries, Israel), in a humidified atmosphere of 5% CO₂ at 37° C. For lentivirus packaging and viral titer determination, 293T and A514 cells were maintained as that of MKN45 and AGS cell lines.

For the selection of CTGF over-expression stable clones, pCDNA3 vector alone and pCDNA3/CTGF plasmid were transiently transfected in MKN45 using Lipofectamine 2000 Transfection reagent (Invitrogen, USA) according to the manufacturer's instructions. pCDNA3/CTGF plasmid was a kind gift from Professor Ming-Liang, Kuo's lab at the Institute of Toxicology of National Taiwan University Medical College. Stable transfectants were selected in Gentamicin (0418; Life Technologies, USA) at a concentration of 600 μg/mL. Thereafter, the selection medium was replaced every 3 days. After 2 weeks of selection in G418, clones of G418 resistant cells were isolated and allowed to proliferate in RPMI1640 containing 200μg/mL 0418. Integration of plasmid DNA was confirmed by reverse transcription-polymerase chain reaction (RT-PCR) and western blot analysis. CTGF stable knockdown clones were generated by co-transfection of packaging plasmids pCMVΔR8.91, pMD.G and pLKO.1-shCTGF (RNAi Core, Academia Sinica, Taipei, Taiwan) into 293T cell line using Lipofectamine 2000 according to manufacturer's instructions. Virus-containing medium was harvested at 24 and 48 hours post transfection of packaging plasmids. AGS cells were grown to confluence and infected using lentivirus-containing medium using polybrene. Stable transfectants were selected in puromycin at a concentration of 8 μg/mL. Thereafter the medium was replaced every 3 days. At 2 weeks after selection, clones of puromycin resistant clones were maintained in 4 μg/mL puromycin. Knockdown was assessed with western blot analysis.

To determine CTGF mRNA expression level of wild type gastric cancer cells and stable CTGF over-expression and knockdown clones, RNA was first purified using TRIzol reagent (Gibco BRL Life Technology, USA.) according to manufacturer's instructions. Briefly, 1 ml of Trizol was added to cell pellets followed by 200 μl chloroform and followed by centrifugation at 14,000 rpm (Kubota, Japan). 500 μl isopropanol (Sigma-Aldrich, USA) was added to the supernatant and centrifuged at 14,000 rpm. The pellet was washed with 75% ethanol and dissolved in DEPC-treated deionized water. RNA quality was analyzed by running 1% agarose gel and concentration determined by NanoDrop (Thenno Fisher Scientific, USA).

For CTGF protein expression level analysis, total protein was isolated by adding mammalian cell lysis buffer (Fermentas Life Science, USA) to the cell pellets. Supernatant was collected after centrifugation at 14,000 rpm, and concentration determined using Bio-Rad protein assay Bio-Rad, USA) and optical density read at 595 nm.

First strand cDNA was synthesized by reverse transcription of total RNA using RevertAid™ H Minus First Strand cDNA Synthesis Kit according to the manufacturer's instructions (Fermentas Life Science, USA). Briefly, 2 μg of total RNA was mixed with oligo d(T) primer and denatured at 65° C. followed by adding buffers, dNTP, RNase and Reverse transcriptase.

The reaction was then incubated at 42° C. for 60 minutes followed by reaction termination at 70□ for 5 minutes.

To determine the CTGF mRNA expression level, RT-PCR was carried out using CTGF specific primers; CTGF forward [5′GCTTACCGA CTGGAAGACACGTT] (SEQ ID NO: 1) and CTGF reverse [5′CATGCCATGTCTCCGTACATC] (SEQ ID NO: 2), RT-PCR products were analyzed by 1% agarose gel electrophoresis.

For CTGF protein expression level in gastric cancer cell lines, 30 μg of total cell lysate were separated in a 10% SDS-PAGE gel and electroblotted onto PVDF membrane (Millipore, USA). The levels of CTGF protein were analyzed on Western blots using anti-CTGF antibody (GTX124232, Genetex, Taiwan) at a concentration of 1:1000, a horse radish peroxidase labeled as secondary antibody (Chemicon, USA) at a concentration of 1:2500, and ECL as chemiluminescent detection reagent (Millipore, USA). Results were exposed onto X-ray film (Kodak) and developed using an automated developer (Kodak X-OMAT 1000 processor).

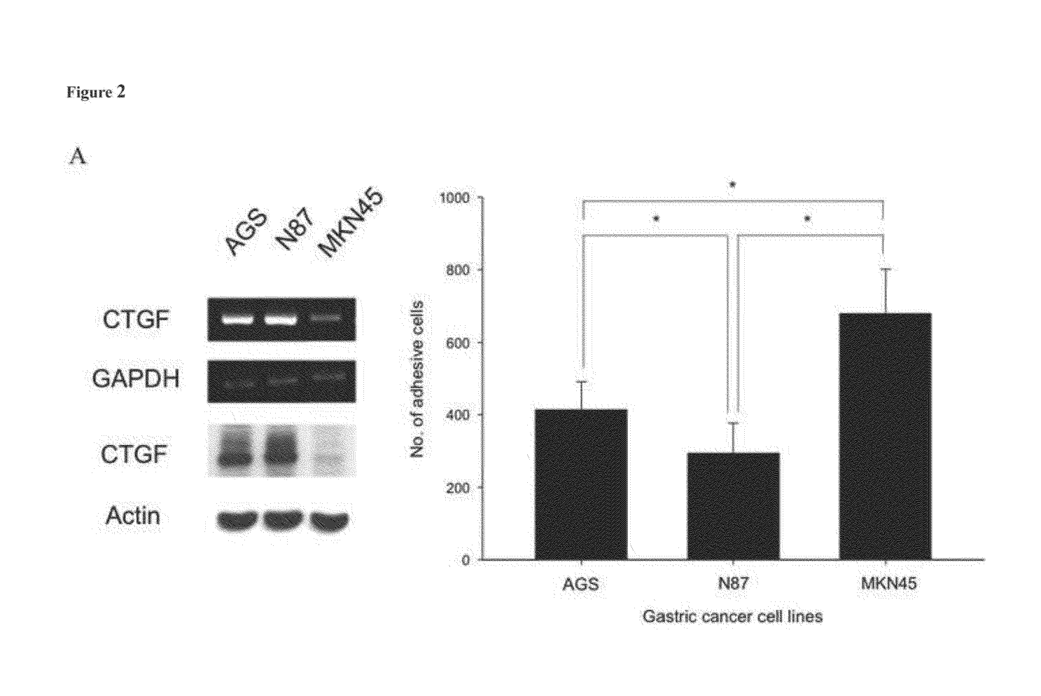

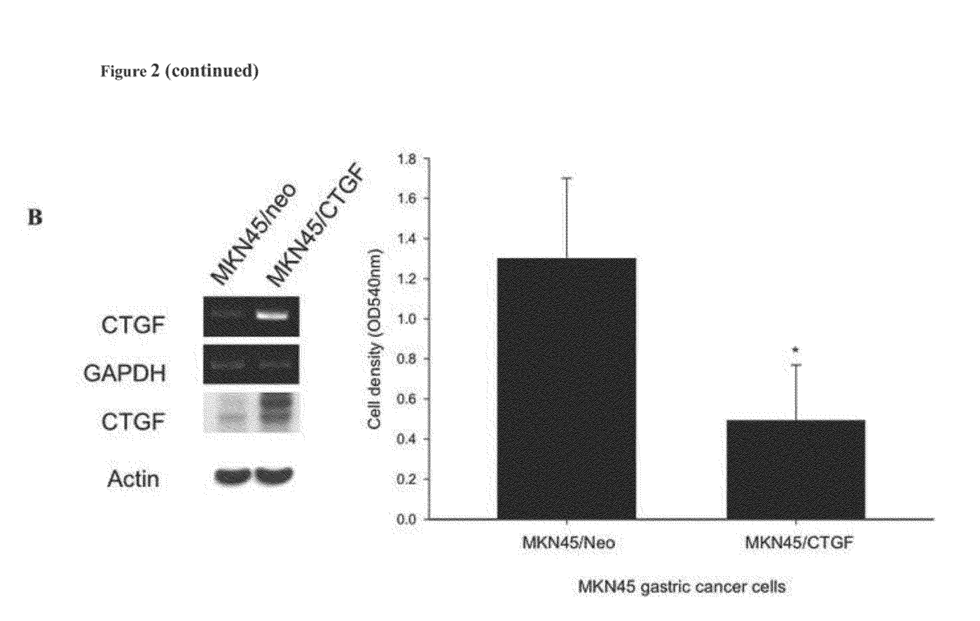

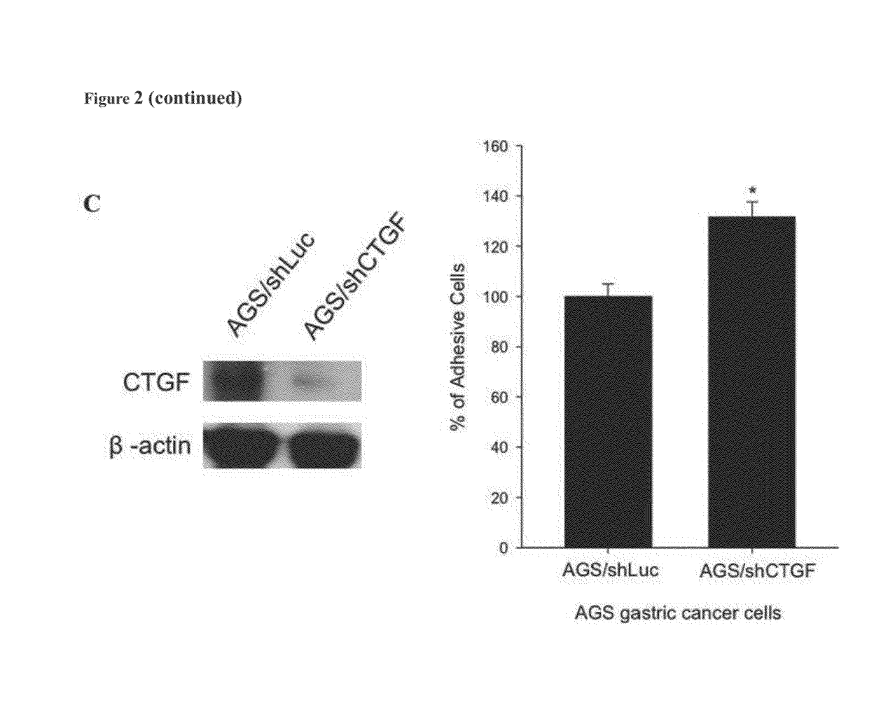

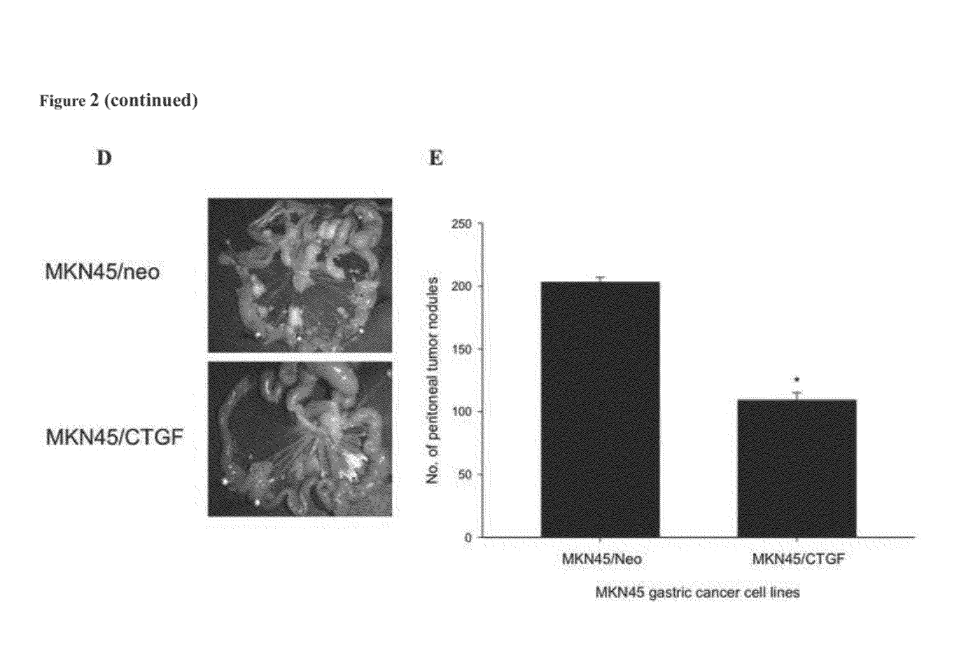

CTGF expression has been determined to be reciprocal to that of calreticulin expression in previous study. It is a gene of interest because of its role as an anti-metastasis protein in colorectal can that is also in gastrointestinal cancer. Therefore to investigate the role of CTGF in peritoneal dissemination in gastric cancer, CTGF expression and in vitro adhesion ability of three different gastric cancer cell lines were determined by western blot and RT-PCR. AGS, N87 and MKN45 were found to have different levels of endogenous CTGF expression (FIG. 2A left panel). N87 had the highest CTGF expression level followed by AGS and lastly MKN45.

To determine the in vitro adhesion ability of wild-type gastric cancer cell lines as well as CTGF over-expressed and knocked-down cells, matrigel (BD biosecience, USA) was coated onto 96 well cell culture plate, cells were counted and seeded at 5000 cells per well, then incubated at 37° C. for 15 minutes. Cells were washed and fixed with 4% paraformaldehyde then stained with 0.058% crystal violet. Number of adhesive cells was counted under a phase contrast microscope.

The adhesion ability of the three cell lines inversely correlated with their CTGF expression level. N87 cells have the lowest adhesion ability and are significantly lower than that of AGS cell lines (P=0.0145) and MKN45 cells (P=0.015). AGS cells also had significantly lower adhesion ability than that of MKN45 cells (P=0.043) (FIG. 2A, right panel). This data confirms the hypothesis and clinical observations that CTGF expression is a factor that determines the adhesion ability of gastric cancer cells. MKN45 was chosen for CTGF over-expression as it is one of the most commonly used gastric cancer cell line and it has comparatively low endogenous CTGF level, thus produces more significant over-expression level. Full-length CTGF eDNA clones were transfected into MKN45 cells and the selection of stable cell line was using 0418 and adhesion ability were tested using in vitro matrigel adhesion assay. When CTGF is over-expressed in MKN45 cells (FIG. 2B, left panel), adhesion to matrigel is decreased by 60% compared to control cell line (P=0.022) (FIG. 2B, right panel). Since MKN45 has lower endogenous CTGF expression, knockdown has little effect on the expression level of CTGF and adhesion ability, therefore AGS cell line has been chosen as a candidate cell for knockdown experiment due to comparatively higher endogenous expression. CTGF was knocked-down using the lentivirus system and adhesion was tested (FIG. 2C, left panel). A 30% increase in adhesion ability was observed in stable CTGF knockdown AGS cell line (FIG. 2C, right panel).

To further confirm the in vitro adhesion assay results, in vivo peritoneal metastasis was carried out in mice model. CB17/Icr-Prkdc^{scid}/Crl mice were purchased from BioLASCO Taiwan, Co., Ltd and housed in microisolator cages and fed autoclaved water and chow ad libitum. All animal work carried out in the animal facilities of the National Taiwan University Hospital adheres to guidelines approved by Institutional Animal Care and Use Committee (IACUC) of National Taiwan University Medical College, Center of Experimental Animals. 2×10⁶ MKN45/Neo or MKN45/CTGF cells, AGS/shLuc or AGS/shCTGF were suspended in PBS and inoculated intra-peritoneusly. Mice were sacrificed and dissected at 45 days after cancer cell inoculation or when moribund. Number of nodules was counted.

To determine if adhesion ability is affected in viva, SCID mice were inoculated with CTGF over-expressed MKN45 and its control cell lines intra-peritoneusly and it was observed that there was a significant decrease in peritoneal nodules in MKN45/CTGF mice (FIG. 2D,E) (P=0.000077). CTGF knockdown clones were also subjected to in vivo peritoneal metastasis experiment using AGS/shCTGF and its control cell line; however significant differences in tumorigenic ability could not be observed (data not shown). This is the result of the comparatively higher endogenous CTGF expression in AGS cells that inhibited control cell line adhesion to the peritoneum, and that insufficient knockdown prevented the cell from aggressively adhering to the peritoneum. However, CTGF over-expression peritoneal metastasis test showed that CTGF plays a significant inhibitory role in adhesion of gastric cancer peritoneal metastasis in vitro and in vivo. The in vivo data also correlates with the in vitro data that CTGF modulates gastric cancer cell adhesion and therefore further supports the potential therapeutic role of CTGF in gastric cancer therapy.

### Example 3

### CTGF Inhibits Gastric Cancer Cell Peritoneal Dissemination Through Blocking Association of Integrin α3β1 to Laminin

To determine the major extracellular matrix involved in the adhesion of gastric cancer cells, collagen type 1, fibronectin, and laminin coated plates were obtained (Millipore, USA). 5×10³ cells were seeded in each ECM substratum and incubated at 37° C. for 1 hour followed by fixing in 4% paraformaldehyde and stained with 0.05% crystal violet. Cells were washed and lysed for determination of optical density at 540 nm. All experiments were done at least three times in triplicates. To further determine the integrin subunits that are involved in the adhesion of gastric cancer cells to laminin, integrin blocking antibodies were obtained from Millipore, USA. 5×10³ cells were blocked by blocking antibodies at 0.5 μg/μl and incubated at 37° C. for 1 hour followed by adhesion assay described above. IgG was used as negative controls in all antibody blocking experiments.

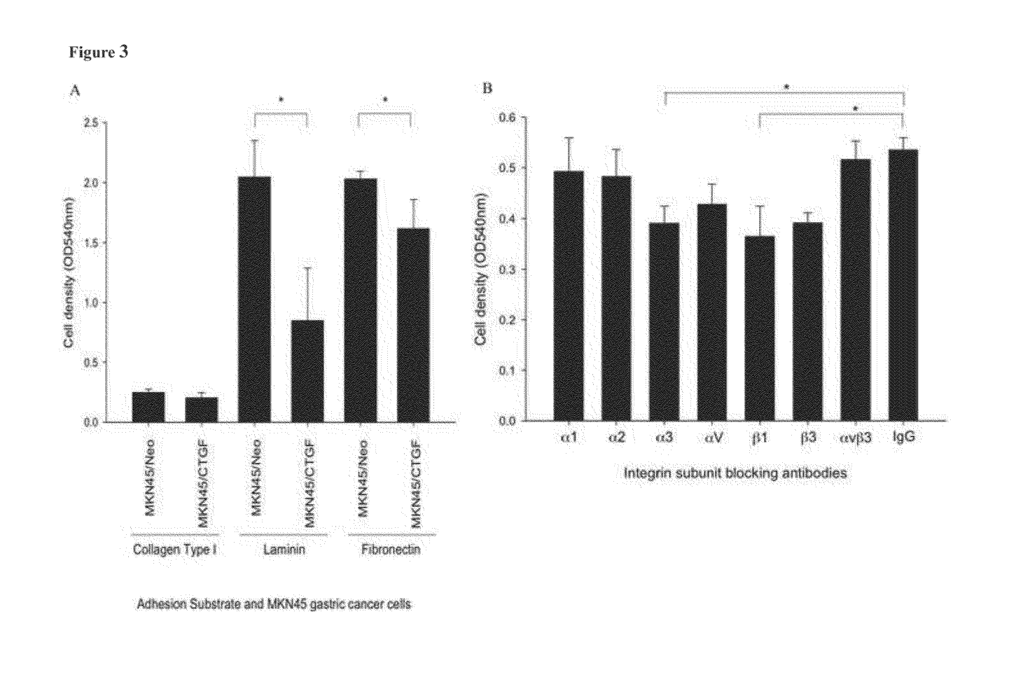

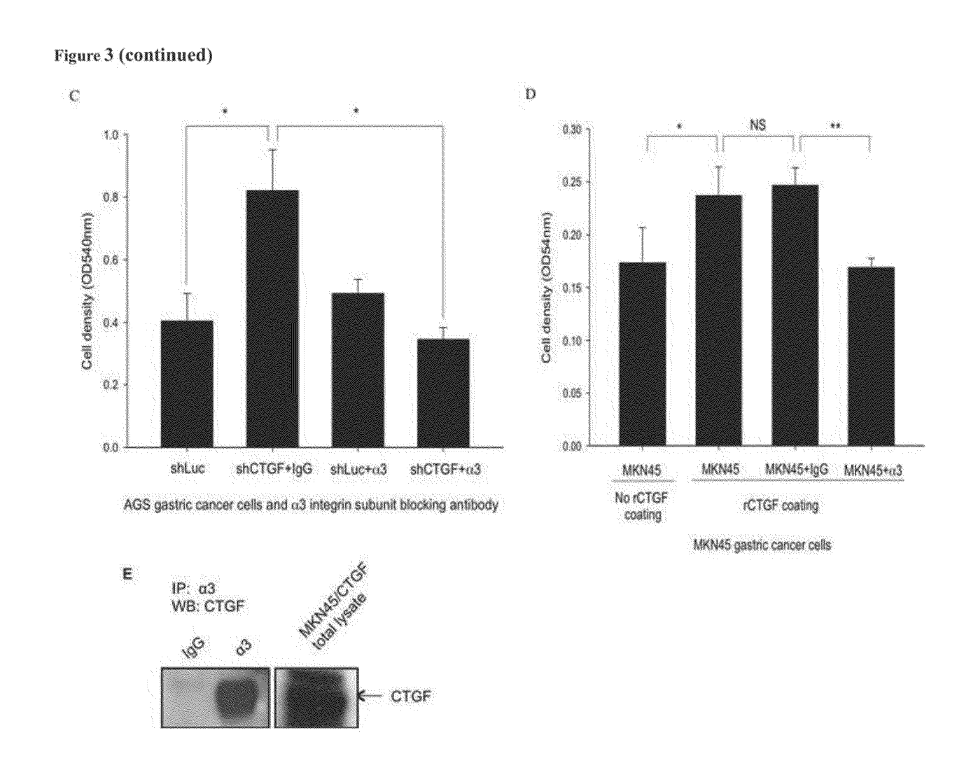

To investigate the molecular mechanism of CTGF induced inhibition of gastric cancer cells to peritoneum, the major extracellular matrix component that is present in matrigel was first screened as a possible adhesion substratum. Adhesion of CTGF over-expressed MKN45 cells to collagen type I, fibronectin and laminin were tested, and CTGF significantly inhibited the adhesion in fibronectin coated and laminin coated culture plate (FIG. 3A). Previous reports have shown that adhesion of gastric cancer cell is mediated through binding of integrin α3β1 to laminin; therefore laminin coated plate has been chosen to further investigate the role of CTGF expression in adhesion.

The integrin subunits involved in CTGF mediated adhesion inhibition in MKN45/CTGF gastric cancer cells were next screened using integrin subunit blocking antibodies. A significant inhibition in adhesion of integrin α3 subunit and β1 subunit antibody blocked MKN45/CTGF cells on laminin coated cell culture plate was observed (FIG. 3B). To further confirm adhesion inhibition, integrin α3 subunit was blocked in CTGF knocked-down AGS gastric cancer cell lines and the adhesion ability was determined. Results showed a significant inhibition in adhesion to laminin coated plate when CTGF-knocked down cells were blocked by blocking integrin α3 blocking antibody (FIG. 3C). Although CTGF was knocked down in AGS cells, adhesion ability was only increased by 30% in previous results; therefore it was speculated that there are still enough CTGF secreted by the cells that can be blocked by integrin α3 blocking antibody and hence the observed adhesion inhibition. In order to confirm the interaction of CTGF and integrin α3 subunit, cell culture plate was coated with a fragment of recombinant CTGF. MKN45 cells that had higher adhesion ability was blocked by integrin α3 subunit and a significant adhesion inhibition to rCTGF coated plate was observed (FIG. 3D). Previous studies have shown that the integrin α3 subunit is involved in the recognition of laminin-5; therefore it was speculated that it is also involved in recognition to CTGF. To further confirm the interaction between integrin α3 and CTGF, co-immunoprecipitation was carried out using the Catch and Release Immunoprecipitation System (Millipore, USA) according to manufacturer's instructions. Briefly, 500 μg of total cell lysate of MKN45/CTGF cells were added to the resin column and as well as 5 μg of anti-integrin α3 subunit monoclonal antibody (Millipore, USA). The reaction was incubated at 4° C. overnight on a rotating wheel and the resin was washed and elution was carried out to obtain immunoprecipitated products. Immunoprecipitated products were subjected to western blot analysis using anti-CTGF polyclonal antibody (Genetex, Taiwan) following the methods mentioned above. Results have shown that CTGF can be detected in anti-integrin α3 pulled-down products (FIG. 3E). The adhesion inhibition induced by CTGF expression was through binding of CTGF to integrin α3β1.

### Example 4

### CTGF is a Potential Therapeutic Agent to Inhibit Peritoneal Dissemination

Previous studies have shown that the C terminal (CT) domain of CTGF protein binds to a variety of members from the integrin family in different cell types, so in order to assess the potential therapeutic effect of recombinant CTGF, in vitro adhesion and in vivo peritoneal metastasis of MKN45 cells were tested under the presence of CT domain of recombinant CTGF (SEQ ID NO: 4).

To determine the therapeutic potential of recombinant CTGF protein, 50, 100 and 200 ng of recombinant CTGF (Peprotech) was added to 1×10⁶ cells and cells were harvested at 6 hours post rCTGF treatment. Harvested cells were subjected to in vitro adhesion assay using matrigel as described above. Dose-dependent rCTGF treated cells were counted under phase contrast microscope to calculate number of surviving cells 6 hours post rCTGF treatment. Each treatment was done in triplicates and p-value was determined by student's t-test.

To confirm the interaction between CTGF and cell surface integrin, 1×10⁴ MKN45 cells were blocked with 0.5 μg/μl anti-integrin α3 subunit antibody and incubated at 37° C. for 1 hour. Cells were then subjected to adhesion assay using rCTGF coated 96 well culture plates that have been coated with 200 ng rCTGF (Genetex, Taiwan). Briefly cells were seeded onto rCTGF coated plates and incubated at 37° C. for 1 hour then washed and stained by 0.05% crystal violet followed by lysis and concentration determined at OD540 nm.

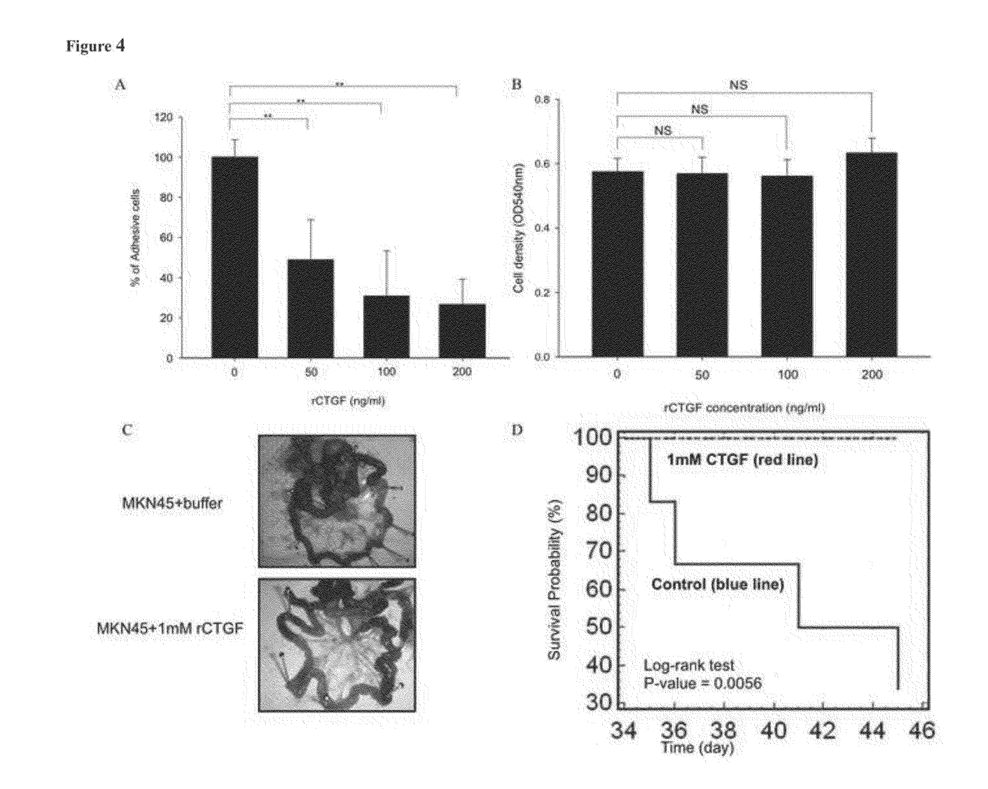

In in vitro adhesion assay, dose dependent adhesion inhibition was observed. At 6 hours post treatment, 50% adhesion inhibition was observed at 50 ng of recombinant CTGF treatment, and 70% and 75% for 100 ng and 200 ng recombinant CTGF respectively (FIG. 4A). To determine if recombinant CTGF poses cytotoxicity, cell density was determined and no significant change in number of cells was observed at 50 ng, 100 ng and 200 ng compared to no recombinant CTGF treatment at 6 hours post treatment (FIG. 4B).

The in vivo therapeutic potential of recombinant CTGF protein has also been determined using mice model, briefly six-week old CB17/Icr-Prkdc^{scid}/Crl mice were co-inoculated with 2×10⁶ MKN45 cells and 0, 0.1 mM or 1 mM rCTGF. At 45 days after co-inoculation, mice were sacrificed and the number of tumor nodules was counted.

To further determine if recombinant CTGF inhibits adhesion in animal model, MKN45 cells and recombinant CTGF were co-inoculated intra-peritoneously, the peritoneal metastasis in SCID mice was observed and the survival probability at 45 days post co-inoculation was calculated. SCID mice were sacrificed and number of tumor nodules were observed at 6 weeks post co-inoculation and buffer control group had more tumor nodule growth than recombinant CTGF treated group can be observed (FIG. 4C) and the buffer control group had significant lower survival probability at 45 days post co-inoculation (p=0.0056) (FIG. 4D). The results have shown that by using recombinant CTGF-CT domain (SEQ ID NO: 4), one can effectively block integrin α3β1, which adds to the integrin family members associated with CTGF as well as demonstrated that only a short domain is required for inhibiting gastric cancer cell to the peritoneum. This suggested that the short sequence allows us to minimize undesired side effects of other modules. In vitro and in vivo data have suggested that a fragment of recombinant CTGF is a potential therapeutic agent to prevent post surgery peritoneal dissemination and perhaps it can be developed as a standard treatment for gastric cancer therapy in the future.

What is claimed is:

1. A method for inhibiting peritoneal metastasis caused by gastric cancer cells in a subject in need thereof comprising administering to the subject a pharmaceutically effective amount of a connective tissue growth factor (CTGF), wherein the CTGF binds to an integrin α3β1 of the gastric cancer cells.

2. The method of claim 1, wherein the CTGF is selected from a CTGF native protein, a CTGF recombinant protein or a C terminal (CT) domain of the CTGF.

3. The method of claim 1, wherein the inhibition of the peritoneal metastasis is due to the binding of the CTGF to the integrin α3β1 of the gastric cancer cells that blocks the gastric cancer cells bind to a laminin of a peritoneal cell.

4. The method of claim 1, wherein the subject is a mammal.

5. The method of claim 4, wherein the mammal is a human.

6. A method for prognosing peritoneal metastasis caused by gastric cancer cells in a subject comprising:
(a) providing a peritoneal tissue from the subject;
(b) measuring a first expression amount of a connective tissue growth factor (CTGF) from the peritoneal tissue; and
(c) comparing the first expression amount to a reference expression amount of the CTOF from a non-peritoneal metastasis gastric cancer tissue,
wherein a decrease in the first expression amount relative to the reference expression amount means the decrease of the CTGT that binds to an integrin α3β1 of the gastric cancer cells and is indicative that the subject is susceptible to peritoneal metastasis.

7. The method of claim 6, wherein the subject is a mammal.

8. The method of claim 7, wherein the mammal is a human.


SEQUENCE LISTING (4 sequences)

SEQ ID NO: 1 - DNA, 23 bp, Artificial
  Synthetic forward primer based on Human CTGF gene
gcttaccgactggaagacacgtt

SEQ ID NO: 2 - DNA, 22 bp, Artificial
  Synthetic reverse primer based on Human CTGF gene
tcatgccatgtctccgtacatc

SEQ ID NO: 3 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 4 - protein, 97 aa, Homo sapiens
GKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA